(19) United States
(12) Patent Application Publication
(10) Pub. No.: US 2014/0296645 A1
(43) Pub. Date: Oct. 2, 2014

(54) LARYNGOSCOPE AND LARYNGOSCOPE BLADES

(71) Applicant: AIRCRAFT MEDICAL LIMITED, Edinburgh (GB)

(72) Inventors: Matthew John Ross McGrath, Edinburgh (GB); Peter Douglas Colin Inglis, Edinburgh (GB); Thomas James Rhodes, Edinburgh (GB)

(21) Appl. No.: 14/358,928

(22) PCT Filed: Nov. 16, 2012
(86) PCT No.: PCT/GB2012/052858

§ 371 Date: May 16, 2014

(30) Foreign Application Priority Data
Nov. 16, 2011 (GB) 1119797.7

(51) Int. Cl. A61B 1/267; A61B 19/00

(52) U.S. Cl. CPC A61B 1/267; A61B 19/44
USPC 600/186; 600/196; 600/193

(57) ABSTRACT

A laryngoscope body for use with laryngoscope blades is presented having an elongate handle and a blade retaining member configured to retain a blade such that the blade extends away from the elongate handle; wherein the blade retaining member is fixable at a plurality of positions to allow a blade to be retained on the blade retaining member at a plurality of—angles relative to the elongate handle. There is also disclosed a kit of parts comprising a laryngoscope body and a plurality of blades, as well as a method of use for the said laryngoscope body with a suitable laryngoscope blade.

### FIELD OF THE INVENTION

The invention relates to the field of laryngoscopes and video laryngoscopes. Some embodiments of the invention relate to video laryngoscopes including an elongate blade retaining member comprising a video camera and configured to slidably receive a removable blade having a cooperating channel in use.

### BACKGROUND TO THE INVENTION

Laryngoscopes are medical devices in common use in oral and tracheal medical procedures, and may be used to obtain view of the glottis or larynx, or to manipulate the tongue, glottis or larynx in order to facilitate insertion of endotracheal tubes or other instruments such as endoscopes, which may be separate pieces of equipment, or may be integral to a laryngoscope.

A laryngoscope typically comprises a body and a blade, which is an elongate section which extends towards and into a patient's oral cavity during a medical procedure such as intubation. A laryngoscope blade may be integrally formed with the body, or demountable. The laryngoscope body typically comprises a handle. Demountable blades can be removed after use and either disposed of or decontaminated for reuse. Single use demountable blades can be particularly useful to avoid cross-contamination.

Some known laryngoscope blades, such as Miller or Wisconsin blades, are substantially flat. However, the blade of a laryngoscope is more commonly bent to better enter through a patient's oropharynx towards their larynx. For example, the most common curvature of a blade is what is commonly called the Macintosh curve. The curvature is relatively gentle and when inserted into a patient's oral cavity, allows a direct view of the patient's larynx in normal situations. Such blades are referred to herein as direct view blades.

However, where there are anomalies within the oral cavity of the patient, or where an effective view of the larynx cannot be achieved with a direct view blade, for example, it is often necessary to use a blade with a greater curvature to allow the blade to be inserted into the oral cavity around any obstructions. In that case, a user cannot normally obtain a direct line of sight view of the larynx and such blades are referred to herein as indirect view blades. In the case of a video laryngoscope, a camera is used to view the region around the trachea during an operation, for example. Video laryngoscopy is especially useful with more curved blades where an indirect view is required.

Currently it is necessary to provide a separate laryngoscope for each situation, or, where disposable or replaceable blades are used, blades must be chosen to suit the situation at hand. Changing blades can be time consuming and the desired blade type may not be available.

Accordingly, it is one object of the invention to provide a laryngoscope that may be effectively used with blades of different curvatures, for example, both direct view blades having a curvature sufficiently gentle to enable a direct view and indirect view blades which have a greater curvature and are configured to obtain an indirect view of the larynx. Some embodiments may also employ moderately curved blades which may be used to obtain either a direct or indirect view.

Some embodiments of the invention address the problem of avoiding unwanted chest interaction which can occur when a laryngoscope is introduced to a patient. For example, when an indirect view blade is introduced into a patient there can be problems with the handle of the laryngoscope contacting the patient's chest.

Additionally, since laryngoscope procedures may require some forceful manipulation of the laryngoscope, it is additionally desirable that the blade and the medical instrument as a whole, be both light weight and mechanically robust. Thus, whereas it is possible to produce robust blades, strength may be at the expense of weight and blade dimensions may require to be larger than optimal. Whereas it has been possible to produce comparatively slimline blades, this has been at the expense of rigidity, durability or suitability for certain procedures and it is known for disposable laryngoscope blades to be either prone to cracking during use, requiring replacement of the disposable portion, or worse resulting in injury or increased risk of infection, or to be sufficiently large as to be difficult to work with. Furthermore, if the blade bends too much under excessive force, the view of the larynx may be compromised.

It is known from EP 1638451 (McGrath) to provide a laryngoscope comprising an elongate member onto which a blade having an elongate channel can be demountably fitted. This elongate member may function as a strengthening element, providing additional mechanical strength to the blade and may comprise a camera and/or light source, for example. However, it is not apparent how such a device could be used with both direct and indirect view blades as if the elongate member was oriented at the correct angle for a direct view blade, one would anticipate that a more curved indirect view blade fitted onto the same elongate member would have its tip in the wrong place.

Accordingly, some aspects of the invention concern laryngoscopes including elongate blade retaining members which are usable with both direct view blades (for example those following the Macintosh curve) and indirect view blades.

Some embodiments of the invention also address the problem that as users of laryngoscopes will often not fit their own blades, some users may not know the type or size of blade fitted to a laryngoscope which is usable with blade of multiple shapes and sizes.

### SUMMARY OF THE INVENTION

Within this specification and the appended claims, the inferior surface is the surface of a laryngoscope blade which faces the patient's tongue in use.

The opposite surface is referred to as the superior surface. Words such as interior, inferiorly, superior and superiorly are used in corresponding senses.

The words distal and distally refer to being towards the end of the blade which extends towards a patient's trachea in use and the words proximal and proximally refer to being towards the person carrying out the medical procedure, in use.

According to a first aspect of the invention there is provided a laryngoscope body comprising an elongate handle and a blade retaining member configured to retain a blade such that the blade extends away from the elongate handle; wherein the orientation of the blade retaining member is variable to allow a blade to be retained on the blade retaining member at a plurality of angles relative to the elongate handle.

Typically, during intubation procedures, a standard fixed laryngoscope is used, which is generally designed for use with a specific laryngoscope blade. Where the intubation procedure requires the use of a blade with a higher curvature, in situations where the patient's airway is obstructed for example, the distal tip of the blade will extend towards the handle of the laryngoscope such that during use the handle will come into contact with the patient's chest. This leads to potential damage to the patient or to the intubation procedure being more difficult to carry out successfully.

The provision of a laryngoscope body having a blade retaining member that can be moved between (and typically fixed at) a plurality of orientations relative to the handle allows the blade retaining member to be oriented at a greater interior angle relative to the handle when using a blade having greater curvature whilst allowing the distal tip of a blade of greater curvature to be sufficiently far from the chest of the patient that intubation can proceed without the handle of the laryngoscope contacting the patient's chest. Therefore, the intubation procedure may be carried out faster or more efficiently than if the laryngoscope itself was required to be changed, and allows the medical practitioner carrying out the procedure to adjust the angle of the blade retaining member as needed.

Preferably, the blade retaining member defines the angle at which a blade retained on the blade retaining member extends from the handle. For example, it might comprise a formation onto which a blade is received (typically, slidably received) to both demountably retain the blade and define the angle at which the blade extends from the handle.

Preferably, the blade retaining member comprises an elongate member for slidably retaining a blade having a cooperating channel. Typically, the elongate member is rotatably mounted on the elongate handle. An elongate member can both retain a blade and define the angle at which a blade extends from the handle. The elongate member is typically rotatable relative to the elongate handle within a plane. Typically, the elongate member is pivotable about a pivot mounting on the elongate handle within a single plane.

The angle between the centre line of the elongate handle and the centre line of the path along which the blade retaining member defines that a blade will extend from the elongate handle is herein referred to as the exit angle. Where the blade retaining member is an elongate member, the exit angle is the angle between the centre line of the elongate handle and the centre line of the elongate member where it extends from the elongate handle. The exit angle is an interior angle during normal operation. In some embodiments, the exit angle may exceed 180 degrees in a non-operating mode.

It may be that the blade retaining member is selectively deployable in a plurality of configurations in which the exit angle is restricted at least to the extent of limiting the maximum exit angle, wherein the maximum exit angle is different in each of the plurality of configurations. As with traditional Macintosh laryngoscopes it may in some circumstances be sufficient to restrict rotational movement of the blade retaining member only to the extent of limiting the maximum exit angle, with the maximum exit angle being different in the different configurations.

It may be that the blade retaining member is selectively fixable in one, or a plurality of positions, that is to say it might be possible to selectively fix the blade retaining member at a particular exit angle or it may be possible to selectively fix the blade retaining member at any of a plurality of different exit angles. The plurality of different exit angles may be discrete or continuous. It may be that blade retaining member can be selectively fixed at one or more discrete exit angles and also be selectively fixed at a range of different exit angles which may or may not overlap with the one or more discrete exit angles.

By allowing a blade to be selectively deployable in a plurality of configurations or selectively fixable in one or more positions, additional flexibility is provided to an operator. For example, where a laryngoscopy procedure requires the use of a blade with a relatively high curvature, such as an indirect view blade, the exit angle can be greater than would be employed with a blade of relatively low curvature, such as a direct view blade. This may for example, avoid the handle of a device using a more highly curved blade from coming into contact with the patient's chest.

The exit angle at which the blade retaining member is selectively fixed, or the maximum exit angle to which the blade retaining member is limited may be selected by a user before a blade is fitted. Nevertheless, the said exit angle or maximum exit angle may be selected during a laryngoscopy procedure (before force is applied) or changed during a laryngoscopy procedure. A manually operable control may be provided to change the exit angle at which the blade retaining member is fixed, or the maximum exit angle to which it is limited. The manually operable control may for example, comprise a manually operable release mechanism or an automatically operable release mechanism.

The manually operable control may comprise a gear or a plurality of gears. In embodiments where the manually operable control comprises a plurality of gears, the plurality of gears may be arranged in a gear train, a worm drive or a planetary arrangement. The manually operable control may provide a resistance to movement of the blade retaining member to provide tactile feedback to the user, assuring the user that the blade retaining member is movable between the secured and locked working positions in a controlled manner.

The manually operable control may comprise a drum mechanism, whereby the drum is fixed to the blade retaining member and mounted to the elongate handle, such that rotation of the drum is coupled to pivoting of the blade retaining member. The manually operable control may comprise a locking mechanism operable to lock the position of the drum.

The locking mechanism may be manually operated by the user by the pushing of a button or pulling a catch, for example. The locking mechanism may be operated via a touch screen and may comprise a solenoid or a motor, for example.

The locking mechanism may comprise a locking pin and the drum may comprise a plurality of apertures configured to receive the locking pin, such that the drum may be locked in a plurality of positions corresponding to a plurality of exit angles of the blade retaining member. The plurality of apertures may comprise one or more apertures configured to receive the locking pin such that it is necessary for the user to retract the locking pin from the said aperture before the drum and blade retaining member can be rotated. The plurality of apertures may comprise one or more apertures configured to receive the locking pin such that the user may rotate the drum and blade retaining member without actively retracting the locking pin.

A manually operable control comprising a drum mechanism may be enclosed within the elongate handle to allow the elongate handle to be sealed and the outer surface to be smooth such that the elongate handle may be readily cleaned or sterilised between uses. In addition, the drum mechanism is able to sustain the torsional forces that are required for difficult laryngoscopy procedures.

The manually operable control may comprise a pair of interlocking plates. Each of the interlocking plates may comprise a series of teeth to reversibly engage the series of teeth of the other plate. The interlocking plates may be rotated relative to each other about a common axis when one of the interlocking plates are moved along the common axis such that the interlocking plates disengage from each other. The blade retaining member may be coupled to one of the interlocking plates, such that rotation of that interlocking plate results in pivoting motion of the blade retaining member. In this way, the blade retaining member may be moved from a first working position (e.g. a position suitable for use with a direct view blade) to a second working position (e.g. a position suitable for sue with an indirect blade), or from a first working position to a storage position, for example.

The manually operable control may comprise a locking mechanism. The locking mechanism may prevent the interlocking plates disengaging. The locking mechanism may comprise a locking pin. The locking pin may be biased towards a position that prevents the interlocking plates disengaging. The locking pin may be retracted manually using a button mechanism, such as a button, for example. The locking pin may be retracted automatically using a solenoid or motor, for example. The pin may be retracted automatically using a touch screen.

A manually operable control comprising interlocking plates separates the positional mechanism from the locking mechanism, allowing each to be engineered separately, and may be enclosed within the elongate handle, allowing the elongate handle to be fully sealed for ease of cleaning and sterilisation between procedures.

The laryngoscope may comprise one or more actuators (e.g. one or more servo motors or stepper motors) to change the exit angle at which the blade retaining member is selectively fixed, or the maximum exit angle to which the blade retaining member is limited. The one or more actuators might be activated by a user operable control (e.g. a button or icon on a touch screen) or automatically, for example responsive to detection of a particular blade type.

By allowing a blade to be used at a plurality of exit angles, it becomes possible for blades with different curvatures to be used with the same laryngoscope while still extending correctly between a patient's teeth through the oropharynx to their larynx, and without unwanted contact with the patient's chest.

For example, the blade retaining member may be selectively fixable in a first position having a first exit angle (or having the maximum exit angle fixed at a first exit angle) suitable for use with a first blade having an inferior surface with a first curvature (for example a direct view blade), and also be selectively fixable at a second position having a second exit angle (or have the maximum exit angle fixed at a second exit angle), which is greater than the first exit angle, suitable for use with a second blade having an inferior surface with a second curvature which is greater than the first curvature (for example an indirect view blade).

It may be that the exit angle (or maximum exit angle as appropriate) is between 120 and 140 degrees in the first position. It may be that the exit angle (or maximum exit angle as appropriate) is between 125 and 135 degrees in the first position. Most preferably, thie exit angle (or maximum exit angle as appropriate) is approximately 130 degrees in the first position.

It may be that the exit angle (or maximum exit angle as appropriate) is between 140 and 180 degrees in the second position. It may be that the exit angle (or maximum exit angle as appropriate) is between 150 and 170 degrees in the second position. It may be that the exit angle (or maximum exit angle as appropriate) is between 155 and 165 degrees in the second position.

It may be that the difference between the exit angle (or maximum exit angle, as appropriate) in the first and second positions is at least 10 degrees, or at least 20 degrees.

It may be that the said first position is a discrete position at which the blade retaining formation may be fixed. It may be that the said second position is a discrete position at which the blade retaining formation may be fixed.

It may be that the exit angle (or maximum exit angle as appropriate) is adjustable to at least, or greater than 180 degrees. It may be that the exit angle (or maximum exit angle as appropriate) cannot be fixed beyond a maximum fixed exit angle. The maximum fixed exit angle may be the said exit angle or maximum exit angle in the second position.

Where the blade retaining member comprises a said elongate member, the elongate member may function as a strengthening element. For example, it may include an elongate strengthening member. The elongate member may therefore be formed from a strong engineered plastics material or metal, or include an elongate strengthening member formed from a strong engineered plastics material or metal.

The advantage of a blade retaining member in the form of an elongate member that functions as a strengthening element that is inserted into the channel of a blade, is that the blades used do not need to be as strong or resilient to deformation than would otherwise be the case. Therefore, the blade may be lighter and/or have a smaller cross-sectional area, making insertion into a patient's oral cavity easier, and the blades cheaper to manufacture.

The elongate handle and the elongate member may lie in a common plane. The axis of the elongate handle and the plane of the elongate member may be parallel, the plane of the elongate handle laterally offset from the plane of the elongate member.

The blade retaining member may have a storage position wherein the blade retaining member is oriented to direct a blade such that the tip of a retained blade extends along, or is partially or fully recessed within the elongate handle. In this position, the exit angle may, for example, be at most 50 degrees.

Typically, the blade retaining member does not retain a blade when in the storage position. It may be that, when in the storage position, the distance between the distal end of the blade retaining member and the elongate handle is less than 5 cm, or less 1 cm. The distal end of the blade retaining member may abut the elongate handle when in the storage position. The distal end of the blade retaining member may be partially or fully recessed within the elongate handle.

In embodiments where the blade retaining member retains a blade when in the storage position, the distance between the distal tip of the retained blade and the elongate handle may be less than 5 cm, or less than 1 cm. The distal tip of the retained blade may abut the elongate handle when in the storage position. The distal end of the retained blade may be partially or fully recessed within the elongate handle.

Thus, the laryngoscope body occupies a more compact volume that is easier to store within a container, for example.

A biasing mechanism may be provided to bias the exit angle between the blade retaining member and the handle. The blade retaining member may be biased to reduce the exit angle. The blade retaining member may be biased to increase the exit angle. The sense of biasing may change with exit angle, for example, the blade retaining member may be biased to reduce the exit angle below a fixed threshold but biased to increase the exit angle above a second threshold angle, so that it can be left at the maximum possible exit angle. Biasing can be advantageous firstly to assist in locating the blade retaining member at certain positions and also in that a user would typically prefer the feeling of operating a blade retaining member which is subject to a biasing force rather than freely moving, which can feel insubstantial.

The blade retaining member may be selectively fixable in two, or three or more discrete positions in which there is a different exit angle between the elongate handle and a blade retained on the blade retaining member.

The blade retaining member may be biased by one or more resilient elements, for example one or more springs.

The laryngoscope may comprise a resilient cover which extends at least over the flexible junction between the handle and the blade retaining member. The resilient cover may take the form of a skin. The resilient cover may extend over the whole of the handle and the blade retaining member. This resilient cover can be useful to prevent contamination in the joint region between the handle and the blade retaining member. The resilient cover may act to bias the blade retaining member, for example to a particular exit angle or range of exit angles, by resisting movement away from the particular exit angle or range of exit angles.

When the blade retaining member is fixed in a position the blade retaining member may be held in place by a locking element, for example a pin. The blade retaining member may be released from the locking element by a release mechanism, for example a button or other user interface element, such as a region of a touch screen (e.g. an icon). The release mechanism may be manually operable, for example it may be located on the elongate handle such that it may be operated by the user, by depression by their thumb, for example, during use. The release mechanism may comprise an automatic mechanism, for example a servo motor actuatable by a user pressing a button, or operating a touch screen.

Preferably, the blade retaining member is pivotally mounted to the elongate handle. The laryngoscope body may comprise a pivot about which the blade retaining member may pivot relative to the elongate handle. Accordingly, the blade retaining member may be pivotally moveable from a first position to a second position, or from a first position to a storage position, for example.

The laryngoscope body may comprise a blade recognition element operable to recognise which type of blade is retained on the blade retaining member. The blade recognition element may be located within the elongate handle of the laryngoscope body. The blade recognition element may be located within the blade retaining member. The blade recognition element may be located within the elongate handle and the blade retaining member. For example, the blade recognition element may be operable to determine whether a blade retained on the blade retaining member is a direct view blade (such as a Macintosh blade) or whether the blade retained on the blade is an indirect view blade.

Blades with a different shape, or blades with equivalent shapes but different sizes, may have different indicators, e.g. different signs, be made of different colours, or have optical or electronically readable devices (e.g. RFID tags) storing different identifier data, or have different packaging.

In embodiments where the blades comprise electronically readable devices, such as RFID tags, for example, the blade recognition element may comprise an aerial and a processor operable to interpret signals received by the aerial and to output a signal dependent on the signal received by the aerial. Thus, the blade recognition element may be an RFID receiver. The aerial may be located within the blade retaining member. The processor may be located within the elongate handle.

Typically, the curvature of a given blade determines whether it is suitable for use with the laryngoscope body when the blade retaining member is in a given position. By curvature of the blade we refer to the change in orientation between the proximal and distal ends of the blade. For example, a blade of a first curvature (for example, a standard blade such as a Macintosh-type blade) suitable for use with the laryngoscope body when the blade retaining member is in the first position may not be suitable, or may be less suitable, for use when the blade retaining member is in the second position. Typically, the curvature of a direct view blade is such that when retained by the blade retaining member in the first position, the distal end of the blade (the end of the blade furthest from the elongate handle) is far enough from the elongate handle that the said elongate handle will not contact a patient's chest during a standard laryngoscopy procedure.

Blades with a higher curvature, such as indirect view blades, for example, require a larger separation between the elongate handle and the distal end of the blade to ensure that the elongate handle does not contact a patient's chest during an laryngoscopy procedure. When such a blade is retained by the blade retaining member in the second position, the greater exit angle ensures that the distal end of the blade and the elongate handle are sufficiently separated that the elongate handle does not contact a patient's chest during a laryngoscopy procedure. Therefore, such blades are suitable for use when the blade retaining member is in the second position, and not suitable for use when the blade retaining member is in the first position.

Therefore, the provision of a blade recognition element ensures that the correct blade type is used in the correct position of the blade retaining member.

The blade recognition element may comprise a Radio Frequency Identification (RFID) reader and the blades to be used with the laryngoscope body may comprise RFID tags. The RFID reader may be operable to read data from a given blade retained on the blade retaining member and to determine whether the correct blade is retained for the current position of the blade retaining member. The laryngoscope body may be configured to (e.g. programmed to) inform the user of the type of the blade retained on the blade retaining member dependent on the determination made by the RFID reader. The type of blade communicated to the user may be chosen from a plurality of types of blade. The plurality of types of blades may comprise direct view blades. The plurality of types may comprise indirect view blades. The plurality of types may include at least two types of blade which have different curvatures which affect how they are operated and are not different sizes of the same type of blades. This is important as blades with significantly different curvatures (e.g. direct view and indirect view blades) are operated quite differently and it is important that a user is aware of the type of blade and to follow the correct procedure.

The laryngoscope body may inform the user as to the procedures suitable for the blade retained on the blade retaining member. The laryngoscope body may inform the user as to whether the blade retained by the blade retaining member is suitable for use in the current position of the blade retaining member. The laryngoscope body may be configured to (e.g. programmed to) convey information to the user via an aural indication, such as an audible alarm when a blade is mounted onto the blade retaining member when the blade retaining member is in the wrong position for that blade, or the type of blade that is mounted onto the blade retaining member, for example. The laryngoscope body may be configured to (e.g. programmed to) convey information to the user via a visual indication. The visual indication may be from one or more light sources, such as light emitting diodes (LEDs). The visual indication may be from a screen. The screen may show the user a video sequence indicating the type of procedure suitable for the blade retained by the blade retaining member, for example.

In the event of the incorrect blade being used for the current position of the blade retaining member, the laryngoscope body may be configured to (e.g. programmed to) warn the user, for example by emitting a sound from a speaker or light from a light source, such as a light emitting diode (LED) or displaying a suitable indication on a screen. In such an event, the laryngoscope body may prevent the user from using the blade by deactivating a screen or light source of the laryngoscope, where the laryngoscope is a video laryngoscope, for example.

The blade recognition element may comprise a cooperating formation and the blades to be used may comprise complementary formations such that the cooperating formation is operable to receive the complimentary formation of one or more correct blades retained on the blade retaining formation or to block the complementary formation of one or more incorrect blades. For example, the cooperating formation may require a blade suitable for use when retained in the first position to comprise a first complementary formation at the blades proximal end, and a blade suitable for use when retained in the second position to comprise a second complementary formation at the blades proximal end.

The blade recognition element may comprise a blocking formation that prevents a blade being retained on the blade retaining member when the blade retaining member is in the wrong position for that particular blade. For example, a blade suitable for use in the second position may be prevented from being retained on the blade retaining member in the first position by the blocking formation.

In this way, a blade suitable for use in the first position would not be able to be retained by the blade retaining member until the blade retaining member is fixed in the first position, or a blade suitable for use in the second position would not be able to be retained by the blade retaining member until the blade retaining member is fixed in the second position, for example.

Accordingly, it would become impossible or difficult to use the incorrect blade for a given position of the blade retaining member, which ensures that the correct blade is used in the correct position for the correct procedure.

Typically, in embodiments comprising an elongate member, the elongate member comprises a proximal end and a distal end, the proximal end adjoining the elongate member to the laryngoscope body and the distal end at the point of the elongate member furthest from the elongate handle.

The laryngoscope body may comprise a display screen assembly. The display screen assembly may extend from the elongate handle. The display screen assembly may be at least partially embedded within the elongate handle. For example, the display screen assembly may be generally oblong (and typically has a major dimension parallel to the length of the handle). One side of the display screen assembly may abut or extend from the elongate handle. In embodiments where the blade retaining member comprises an elongate member, the display screen assembly may extend from the elongate handle in a plane normal to the common plane within which the elongate handle and the elongate member lie.

The display screen assembly may comprise a display screen. Typically, the display screen has an inner edge and an outer edge, and the inner edge is within the lateral extent of the elongate handle and the outer edge extends beyond the lateral extent of the elongate handle.

The display screen assembly may have a first end and a second end, the second end being closer to the blade retaining member than the first end; the laryngoscope body may comprise a grip portion extending from the second end of the display screen assembly to the proximal end of the blade retaining member; wherein the distance between the second end and the proximal end is between 3 cm and 9 cm. Accordingly, the resulting laryngoscope body is compact and can be comfortably held by the user.

The display screen may be a touch screen such that the image received and displayed by the display screen assembly may be manipulated by the user's fingers or thumb whilst being viewed. The brightness or contrast of the image may be altered by pressing or touching controls displayed on the screen, for example.

In this way, the user may operate the touch screen with their thumb, for example, without needing to adjust or change the user's gripping position on the grip portion of the laryngoscope body. Accordingly, the touch screen can be easily and quickly operated during a procedure without the user compromising their grip on the laryngoscope body.

In embodiments where the laryngoscope body comprises actuators to change the exit angle of the blade retaining member, the display screen may be used to select or change the exit angle (or the maximum exit angle, as appropriate) of the blade retaining member.

The display screen assembly may have a thickness of less than 5 mm to avoid bulk and to keep the centre of graining towards the blade. The display screen assembly may be located on the front (user facing) surface of the body.

The display screen assembly may be integral to the body, or may be connected to the body. In embodiments comprising an elongate blade retaining member, the display screen assembly may be pivotable about an axis extending generally perpendicular to a median plane within which the elongate handle and the elongate member lie. The display screen may be pivotable about a pivot extending from the body, generally perpendicular to the median plane. The display screen may be flexible.

The display screen is advantageously large, so as to provide the most detailed images to the user. The display screen may comprise all or the majority of the face of the display screen assembly viewable to a user of the laryngoscope during a medical procedure. The display screen preferably has a diagonal dimension greater than 2.5 cm (1 inches), of greater than 5 cm (2 inches), or at least 6 cm (2.4 inches) or at least 8 cm (3.1 inches). Preferably the diagonal dimension is less than 13 cm (5 inches), less than 12 cm (4.7 inches) or less than 10 cm (3.9 inches). In some embodiments the diagonal dimensions is between 8.5-9.0 cm (around 3.5 inches) or 10.0-11.0 cm (around 4.3 inches).

The aspect ratio is typically between 1:1 (height:width) and 2:1 (height:width), more typically between 1.2:1 and 1.6:1, for example around 1.4:1. The surface area is typically at least 15 cm² and preferably at least 20 cm². The surface area is typically less than 100 cm² and preferably less than 60 cm² or less than 40 cm².

Another advantage of the variable exit angle is that this can enable a user to vary the angle between the blade and the screen by adjusting the screen orientation or the exit angle in use in order to obtain a better view of the screen.

Preferably, the blade retaining member comprises a camera. The camera may be located at the distal end of the blade retaining member. In use, the blade may be situated such that a view of the larynx of the patient may be obtained via the camera. In embodiments comprising a display screen assembly, the camera may be connected to the display screen assembly such that an image of the patient's larynx obtained by the camera may be displayed on the display screen assembly.

The blade retaining member may comprise a light source. The light source may be located adjacent to the camera such that light emitted by the light source reflects from a target to be detected by the camera.

In embodiments comprising a camera, a light source and/or a display screen assembly, the elongate handle may comprise a power source, such as a battery, to provide power to the camera, light source and/or display screen assembly.

The laryngoscope body may comprise a force meter operable to measure the force applied by the operator to the blade retaining member via the blade during a laryngoscopy procedure. The force meter may warn the operator if the force applied to the blade exceeds a pre-set limit. The warning may be the emission of a sound, or the lighting of a light source, such as a light emitting diode (LED), or the display of a warning on a screen, for example. The force meter may release the position of the blade retaining member when a second pre-set force is exceeded to prevent the blade causing damage or further damage to the patient.

The laryngoscope body may be configured to indicate to a user whether a blade retained by the blade retaining member is suitable for use in the current position of the blade retaining member upon recognition of the retained blade by the blade recognition element.

The laryngoscope body may be configured to prevent use of a retained blade that is not suitable for use in the current position of the blade retaining member upon recognition of the retained blade by the blade recognition element.

The blade retaining member may be movable automatically from a first position to a second position automatically.

According to a second aspect of the invention there is provided a laryngoscope blade comprising a mounting element for mounting the laryngoscope blade to a blade retaining member and an electronically readable storage medium operable to store information relating to the type of the laryngoscope blade wherein the electronically readable storage medium is readable by the blade recognition element of the laryngoscope body according to the first aspect of the invention.

The electronically readable storage medium may be a radio frequency identification tag. The electronically readable storage medium may be random access memory (RAM). The electronically readable storage medium may be read-only memory (ROM).

The information relating to the type of the laryngoscope blade may be indicative of to the curvature of the inferior surface of the laryngoscope blade. The information relating to the type of the laryngoscope blade may be indicative of the size of the laryngoscope blade.

Preferably, the mounting element is an elongate channel. The elongate channel typically cooperates with the mounting element.

The invention extends in a third aspect to a kit of parts comprising a laryngoscope body according to the first aspect, and a plurality of laryngoscope blades according to the second aspect, wherein the plurality of laryngoscope blades comprises at least one laryngoscope blade having an inferior surface with a first curvature, and at least one laryngoscope blade having an inferior surface with a second curvature which is different to the first curvature.

The at least one laryngoscope blade having an inferior surface with a first curvature may be a direct view blade (i.e. suitable for use to directly view the larynx of a patient). The at least one laryngoscope blade having an inferior surface with a second curvature which is different to the first curvature may be an indirect view blade (i.e. suitable for use with difficult airways to indirectly view the larynx of a patient). The at least one laryngoscope blade having an inferior surface with a second curvature may not be suitable for use to directly view the larynx of a patient.

The first curvature may differ from the second curvature (each defined as the change in orientation between the proximal and distal ends of the blade) by at least 10 degrees.

According to a fourth aspect of the invention there is provided a laryngoscope comprising a laryngoscope body according to the first aspect of the invention, wherein the blade retaining member retains a blade, the blade extending away from the elongate handle, wherein the orientation of the blade retaining member is variable to vary the exit angle between the blade retaining member and the handle.

The invention extends in a fifth aspect to apparatus comprising a laryngoscope body according to the first aspect of the invention and a plurality of blades, each blade in the plurality of blades comprising a mounting element for mounting the laryngoscope blade to the blade retaining member, the plurality of blades comprising a first blade having an inferior surface with a first curvature (for example, a direct view blade), and a second blade having an inferior surface with a second curvature which is different to the first curvature (for example an indirect view blade). The apparatus may be provided in the form of a kit.

Preferably, the blade retaining member comprises an elongate member for slidably retaining a blade having a cooperating channel. The elongate member may be fixable at a plurality of angles relative to the elongate handle. Further optional features of the elongate member are discussed above in relation to the first four aspects of the invention. For example, the elongate member may function as a strengthening element.

Preferably, each of the blades within the plurality of blades comprise a said channel to receive the blade retaining member; the plurality of blades comprises two blades and the inferior surface of each of the two blades has a different curvature adjacent to the channel of each blade. By curvature adjacent to the channel of the blade, we refer to the change in orientation of the inferior surface between the distal and proximal ends of the channel. For example, a first blade within the plurality of blades may have a first curvature, and a second blade within the plurality of blades may have a second curvature. The first curvature may be greater than the second curvature or vice versa. Typically, the curvature of the elongate member (and the channel of each blade) is the same as the curvature of the inferior surface of the blade with the most curved inferior surface adjacent the channel.

Preferably, the channel of each blade within the plurality of blades has the same curvature, and corresponds to the curvature of the blade retaining member of the laryngoscope body.

However, it may be that two blades in the plurality of blades have channels with different cross-sections. It may be that the external shape of two blades in the plurality of blades differs by more than scale.

Preferably, each blade within the plurality of blades comprises a lifter portion at the distal end of the blade; the plurality of blades comprises a first blade and a second blade wherein the first and second blades have lifter portions extending in different orientations.

In this application, the term lifter portion refers to that portion of the blade that extends from the distal tip of the blade to the distal end of the channel. Typically, a first blade having a greater curvature adjacent to the channel of the blade than a second blade will have a litter portion oriented at a greater angle from the tangent of the end of the channel.

Alternatively, a first blade may have the same curvature adjacent to the channel of the blade as a second blade, but the lifter portion of the first blade extend at a different angle than that of the second blade.

The curvature of the inferior surface adjacent to the channel of each blade within the plurality of blades may vary between each blade.

Preferably, the blade retaining member of the laryngoscope body has a curvature equal to the curvature of the inferior surface of the blade with the greatest curvature within the plurality of blades.

The apparatus may be a kit. The apparatus may be a storage facility. The apparatus may be an operating theatre. The apparatus may be a hospital.

According to an eighth aspect of the invention there is provided a method of using a laryngoscope comprising the steps of;
providing a laryngoscope body according to the first aspect of the invention, a first blade and a second blade;
fixing the blade retaining formation in a first position having a first exit angle or deploying the blade retaining formation in a first configuration in which the exit angle is limited to a first maximum exit angle;
mounting the first blade to the blade retaining formation of the laryngoscope body;
using the laryngoscope body and first blade;
demounting the first blade;
fixing the blade retaining formation in a second position having a second exit angle which is different to the first exit angle, or deploying the blade retaining formation in a second configuration in which the exit angle is limited to a second maximum exit angle, which is different to the first maximum exit angle;
mounting the second blade to the blade retaining formation; and
   using the laryngoscope body and second blade.

The step of fixing the blade retaining formation in a first position having a first exit angle or deploying the blade retaining formation in a first configuration in which in which the exit angle is limited to a first maximum exit angle, and the step of mounting the first blade to the blade retaining formation may take place in either order.

The step of fixing the blade retaining formation in a second position having a second exit angle or deploying the blade retaining formation in a second configuration in which in which the exit angle is limited to a second maximum exit angle, and the step of mounting the second blade to the blade retaining formation may take place in either order.

Preferably, the curvature of the inferior surface of the first blade adjacent to the channel of the blade is different to the curvature of the inferior surface of the second blade adjacent to the channel of the blade.

Preferably, the method comprises the step of choosing between the first and second blade based upon the curvature of the inferior surface adjacent to the channel of each blade.

Typically, laryngoscopes have a fixed blade or detachable blades that are suitable for use with a particular laryngoscope. For example, a first laryngoscope may be designed for use in standard intubation procedures and standard blades, such as Macintosh blades, are suitable for use with the first laryngoscope. During the procedure it may become apparent that the patient's airway is obstructed, and that it is necessary to use a blade with a higher curvature such as an indirect view blade. Such blades are generally not suitable for use with a standard laryngoscope, due to the proximity of the distal tip of a mounted blade to the handle of the standard laryngoscope. Therefore, a second laryngoscope is required to use these blades with high curvature and the laryngoscope must be changed during the procedure.

Changing the laryngoscope during an intubation procedure is time consuming, the required second type of laryngoscope may not be available and the need to have two laryngoscopes for a single procedure is inefficient and expensive. Therefore, the provision for a method of use of the laryngoscope body of the first aspect of the invention where the same laryngoscope body can be used during a procedure where an obstructed airway is discovered with either of the two types of blade (direct and indirect view) is more efficient, the equipment costs are lower and allows the procedure to be carried out faster, potentially saving a patient's life.

The invention extends to a eighth aspect there is provided a method of using a laryngoscope comprising the steps of:
providing a laryngoscope body according to the first aspect and a blade configured to be retained by the blade retaining member;
fixing the blade retaining member of the laryngoscope body in a first position having a first exit angle or deploying the blade retaining member in a first configuration having a first maximum exit angle;
mounting the blade to the blade retaining member of the laryngoscope body;
inserting the blade into the oral cavity of a patient;
fixing the blade retaining member of the laryngoscope body to a second position having a second exit angle which is different to the first exit angle or deploying the blade retaining member in a second configuration having a second maximum exit angle; and
   viewing the oral cavity, or a portion of the oral cavity of the patient.

During an intubation procedure, it may become apparent that it would be preferable to use an indirect view blade which is more curved than a direct view blade. Using laryngoscopes known in the art, it would be necessary to withdraw the inserted blade from the patient's oral cavity, and use a second laryngoscope adapted for use in these situations. This is time consuming and requires a second laryngoscope to be present for any given intubation procedure.

Therefore, the ability to adjust the position of the blade retaining member from a first position to a second position, whilst the blade mounted onto the blade retaining member is inserted into a patient's oral cavity allows the intubation procedure to be carried out much more efficiently and with a lower equipment cost than is possible with laryngoscopes in the art.

Optional features discussed in relation to any aspect of the invention are optional features of each aspect of the invention.

### DESCRIPTION OF THE DRAWINGS

An example embodiment of the present invention will now be illustrated with reference to the following Figures in which:

FIG. 1 is a side view of a laryngoscope body with a blade having a first curvature, and a side view of a laryngoscope body with a blade having a second curvature;

FIG. 2 is a side view of a laryngoscope body where the blade retaining member is in a first position and a view of a laryngoscope body where the blade retaining member is in a second position without (A) and with (B) suitable blades retained;

FIG. 3 is a side view of a laryngoscope body showing the change in angle of the proximal and distal ends of a retained blade in the first and second positions;

FIG. 4 is a perspective view of a series of laryngoscope bodies having a blade retaining member in a first position, a second position, a further position in which the blade cannot be fixed and a storage position;

FIG. 5 is an exploded perspective view of a laryngoscope body showing a fixing mechanism for a blade retaining member having two positions in which the blade can be fixed;

FIG. 6 is a section view of the fixing mechanism showing how the blade retaining member is moved from the second position to the first position;

FIG. 7 is an exploded perspective view of a laryngoscope body showing a manually operable control mechanism for a blade retaining member having four positions in which the blade can be fixed;

FIG. 8 is series of perspective views of the fixing mechanism of FIG. 7 showing the positions in which the blade retaining member may be fixed;

FIG. 9 is an exploded perspective view of a laryngoscope body showing a manual operable control interlocking plate mechanism;

FIG. 10 is an illustration of how the position of the blade retaining member of FIG. 9 may be altered; and

FIG. 11 is a perspective view showing the available range of movement of the blade retaining member relative to the laryngoscope body in example embodiments.

### DETAILED DESCRIPTION OF AN EXAMPLE EMBODIMENT

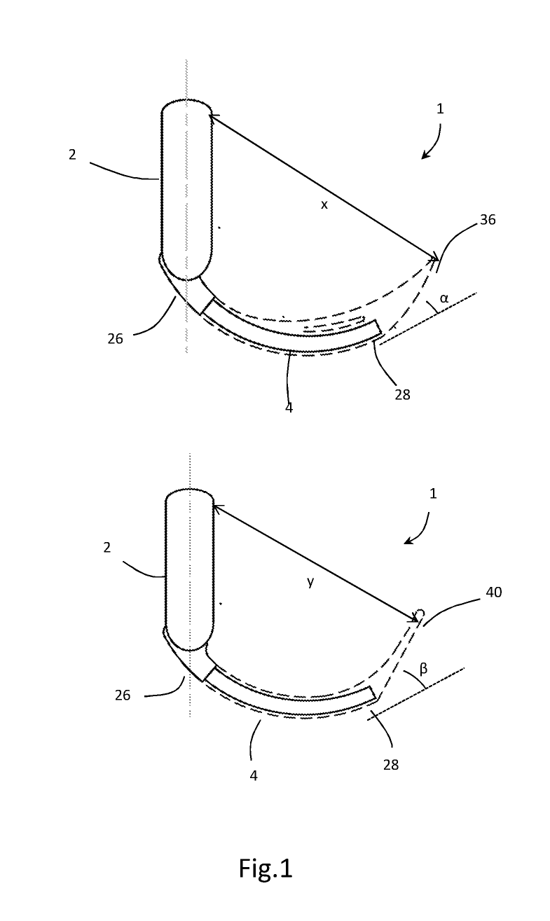

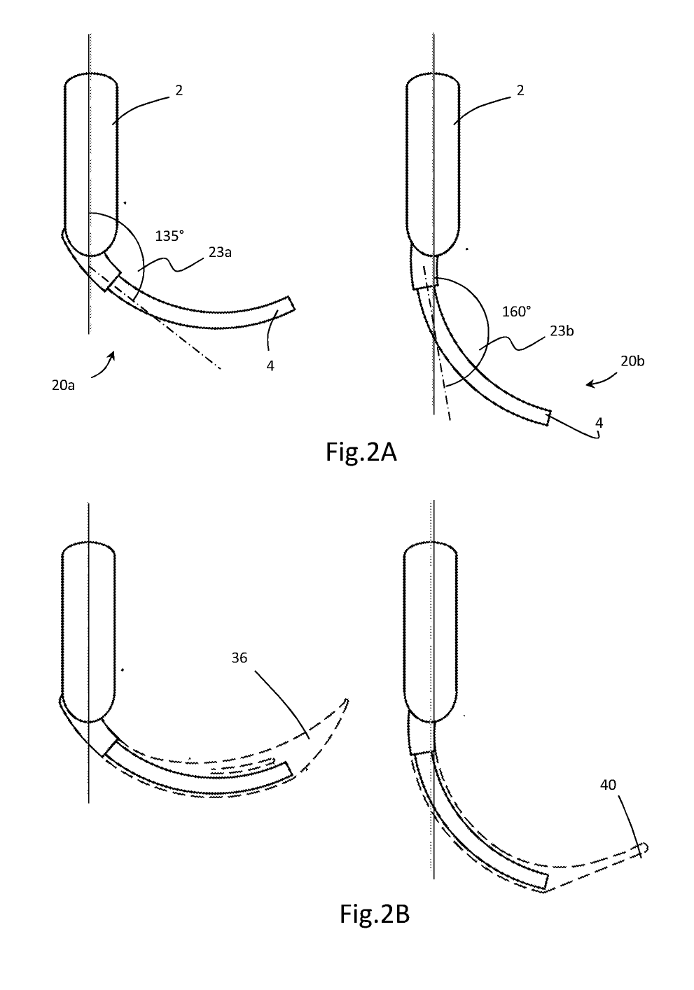

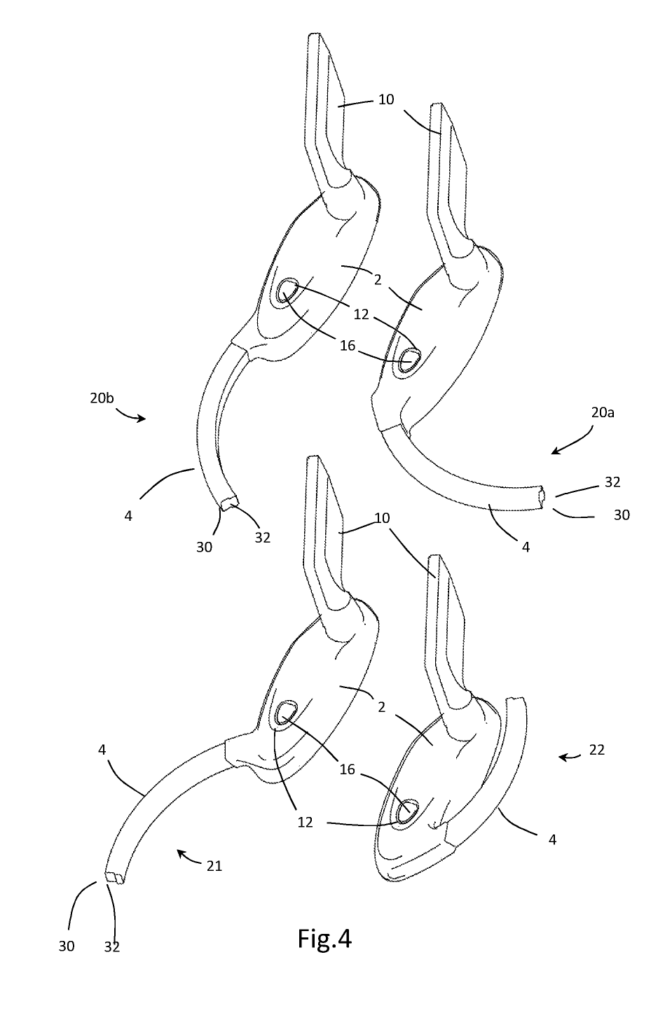

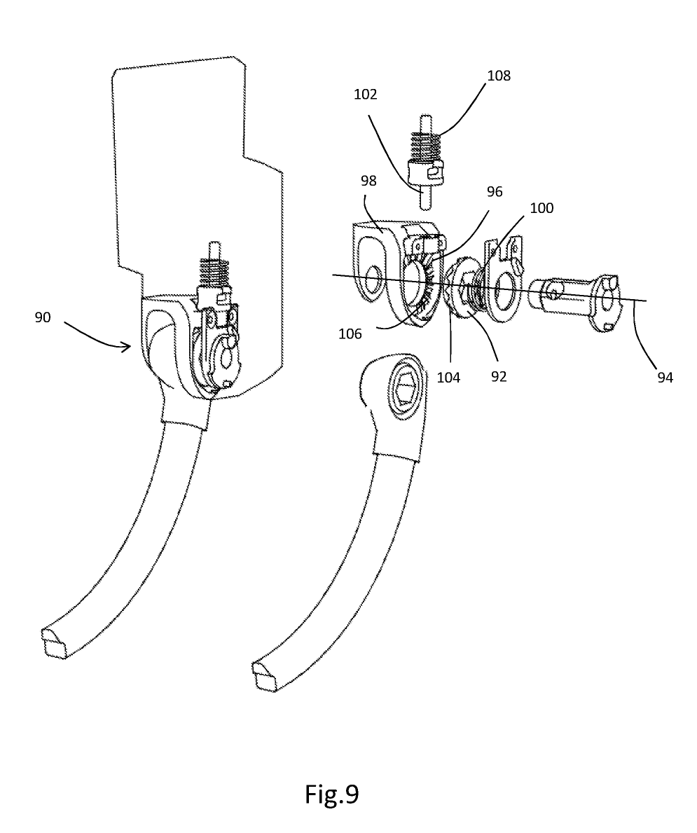

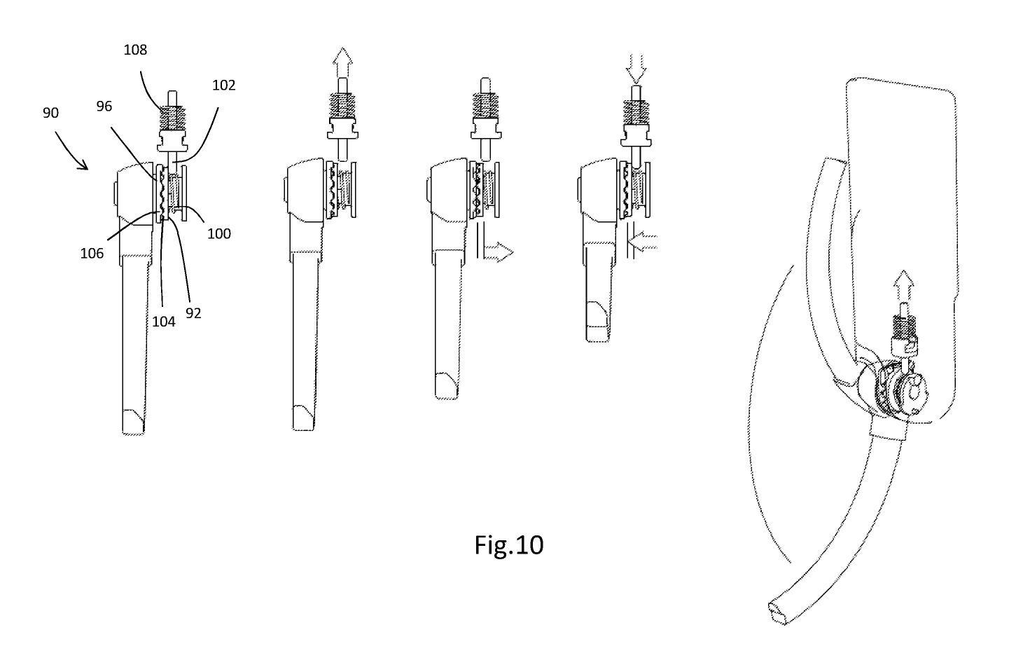

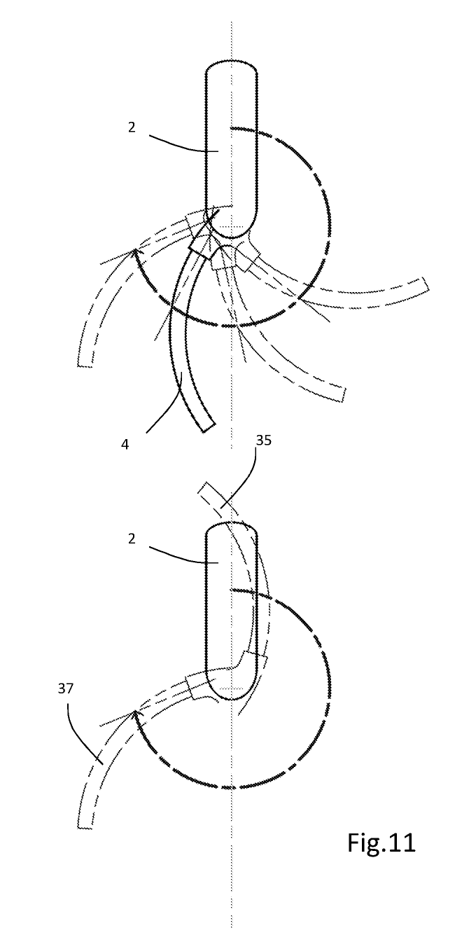

With reference to FIGS. 1 to 11 a laryngoscope body 1 comprises a handle 2 (functioning as the elongate handle) and a blade retaining member 4 pivotally mounted to the handle. The handle has a first end 6 and a second end 8 and comprises a display screen 10 adjacent to the first end (acting as a display screen assembly), an adjuster 12 adjacent to the second end, and a battery 14.

The adjuster comprises a release button 16, four guides (not shown) that define two discrete positions 20a, 20b in which the blade retaining member can be fixed, a position 21 in which the blade retaining member cannot be fixed and a storage position 22 where blade retaining member can be fixed, and a spring (acting as a resilient element). In the first position, the blade retaining member is fixed at an exit angle of 135°relative to the handle. In the second position, the blade retaining member is fixed at an exit angle of 160°relative to the handle. The blade retaining member can also be pivoted to extend to an exit angle of up to 190°relative to the handle but cannot be fixed in that position. The storage position fixes the blade retaining member such that it lies along the handle in a compact form suitable for storage, with an exit angle of around 30 to 40°.

The four guides are defined by a series of partitions, each guide separated from the adjacent guide by a partition. For example, the first position is separated from the storage position by a first partition and from the second position by a second partition. The partitions may be lowered when the release button is depressed such that the blade retaining member may be pivoted between a first position to a second position (either positions or the storage position).

The spring biases the blade retaining member from the maximum possible exit angle towards the second position, and from the storage position towards the first position.

The blade retaining member is an elongate tube of constant curvature and extends from the handle at its proximal end 26 to its distal end 28. The elongate tube of the blade retaining member is sufficiently resilient to deformation in the plane of the curve of the elongate tube that it acts as a strengthening element for any blade mounted onto the blade retaining member. The blade retaining member comprises a camera 30 and a light emitting diode 32 (LED, acting as a light source) at its distal end, and a clip 34, to ensure that a blade mounted onto the blade retaining member is retained.

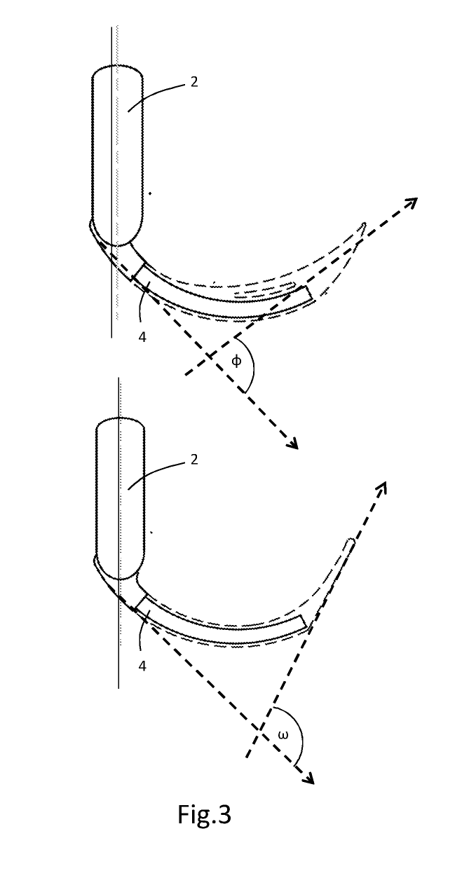

The curvature of blades retained on the blade retaining member can be measured by the change in angle from the proximal end of the blade (adjacent to the elongate handle when retained) to the distal tip of the blade. For example, as shown in FIG. 3, the change in angle for a direct view blade (defined by the angle φ) is smaller than that for an indirect view blade (defined by the angle ω), therefore indicating that a standard blade has a lesser curvature than an indirect view blade. In this example, the different blades all have inferior surfaces with different curvatures in the region adjacent the blade retaining member, as well as different overall curvatures.

As shown in FIG. 1, when the blade retaining member is in the first position, the distal tip of a standard blade retained on the blade retaining member is a distance x from the handle to the laryngoscope body. If an indirect view blade is retained on the blade retaining member in the first position, the distance between the handle and the distal tip of the blade is reduced to y and may result in the handle contacting the patient's chest during an intubation procedure.

The display screen, LED and camera are powered by the battery in the handle.

The handle further comprises a force meter adjacent to the second end abutting the blade retaining member. The force meter measures the force applied to the blade retaining member and sounds an alarm or displays a warning when a threshold force is exceeded.

When the laryngoscope body is not in use, the blade retaining member is fixed in the storage position 35 resulting in a more compact shape that may be stored with a more efficient use of space.

Before use in an intubation procedure, the release button of the adjuster is depressed and the blade retaining member is pivoted from the storage position to the first position. For standard intubation procedures, a Macintosh-type (standard) blade 36 having a first curvature of the inferior surface comprising a channel 38 having the same internal curvature as the blade retaining member, is mounted onto the blade retaining member by inserting the blade retaining member into the channel of the blade and securing the clip of the blade to the blade retaining member. The channel has a transparent window at its distal end to allow the camera of the blade retaining member to receive light. The distal tip of the blade is oriented relative to the line of sight of the camera within the blade retaining member at an angle α.

The blade is then inserted into the oral cavity of a patient, the LED, camera and display screen are turned on and the operator attempts to obtain a view of the patient's larynx. In standard procedures, the LED illuminates the patient's larynx and the camera captures an image of the larynx and transmits the image to the display screen on the handle of the laryngoscope body, to be viewed by the operator. In the event of an obstruction in the patient's oral cavity preventing a view of the larynx being readily obtained, two alternate courses of action may be taken.

The first option is for the operator to apply a greater pressure to the blade to force the obstructing anatomy to be physically moved out of the line of sight. If the handle of the laryngoscope body contacts the chest of the patient, the blade retaining member can be moved from the first position to the second position by depressing the release button and moving the blade retaining member appropriately. The second position provides a greater leverage for the operator to apply greater force. This approach risks damaging the tissue of the oral cavity and teeth of the patient, and could cause the blade to break in situ if the blade has any manufacturing defects or if the applied force is too great. Accordingly, the force meter within the handle of the laryngoscope body warns the operator by sounding an alarm that the force applied exceeds a predetermined threshold and that the patient may be injured.

The second option is for the operator to remove the blade from the oral cavity of the patient and to remove the blade from the blade retaining member for disposal. The blade retaining member is moved from the first position to the second position by depression of the release button as outlined above. A second blade is then fitted to the blade retaining member as per the first blade, having a greater curvature of the inferior surface such as an indirect view blade 40, but with a channel having the same curvature as that of the blade retaining member. The distal tip of the blade is oriented relative to the line of sight of the camera within the blade retaining member by the angle β, where β is greater than α. The second blade is then inserted into the patient's oral cavity and the combination of the greater curvature of the blade and the greater exit angle in the second position allow the operator to manipulate the patient's soft tissue in the oral cavity to obtain a view of the patient's larynx.

Procedures where the patient's oral cavity is obstructed, using instruments known in the art, typically require changing the laryngoscope body itself, as each laryngoscope body is designed for use with a single blade type. This can be a time consuming process and relies on two laryngoscope bodies being available for any given intubation procedure, increasing costs. However, the present invention allows a single laryngoscope to be used with both a Macintosh-like direct view blade and an indirect view blade provided that a stock of both types of blade is maintained. Often a practitioner will choose between the two blade types before the procedure.

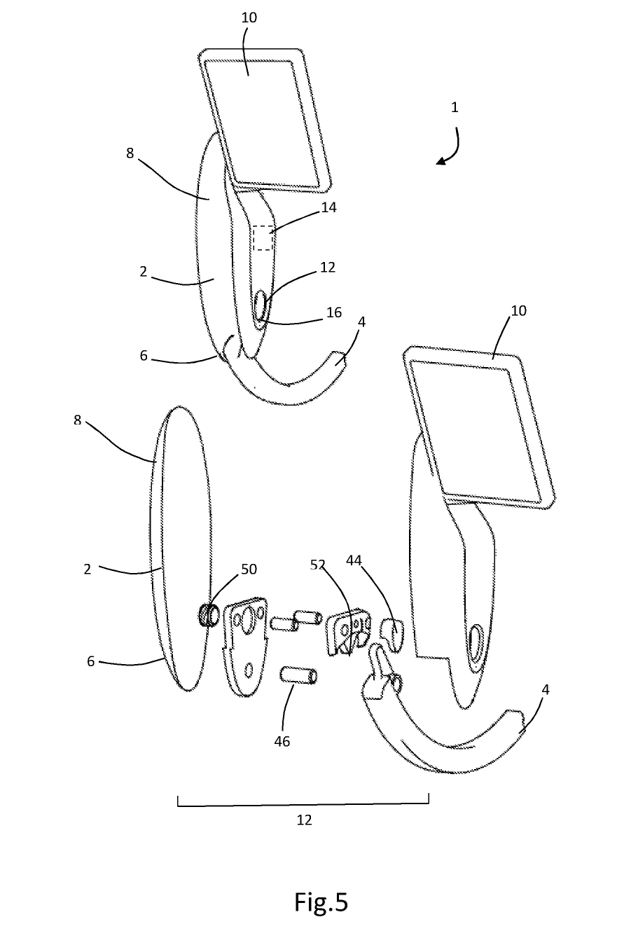

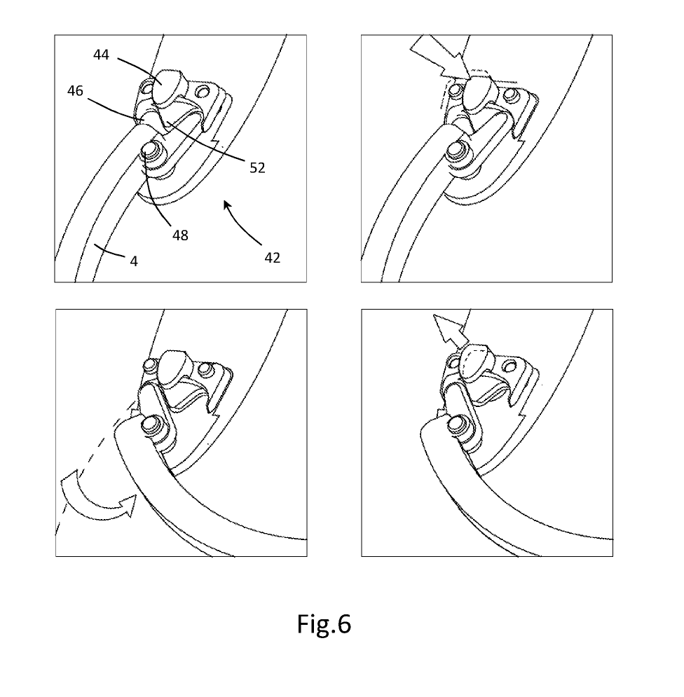

In an alternative embodiment the blade retaining member of the laryngoscope body has two positions, a first position and a second position, as shown in FIGS. 5 and 6, where the adjuster mechanism 42 comprises a release button 44 connected to a plate 46 and a pivot 48, the plate is mounted on a spring 50 and comprises a partition 52. The proximal end of the blade retaining member is mounted onto the pivot such that motion about the pivot is obstructed by the partition. Depression of the release button pushes the plate down against the spring and thereby lowers the partition such that the blade retaining member may freely pivot about the partition from a first position to a second position, for example.

The blade retaining member may be movable from the first position to the second position by way of an automatic mechanism comprising one or more servo motors. A locking pin may be automatically retracted and then redeployed after movement. The blade retaining member may move to a particular position responsive to a user input, for example pressing a button or touching a user interaction element (e.g. icon) on the screen.

The user may touch a user interaction element (e.g. icon) on the screen using their thumb, whilst retaining their grip on the elongate handle. Accordingly, the user may change or adjust the exit angle of the blade retaining member without altering their grip on the elongate handle, thereby minimising disruption of the intubation.

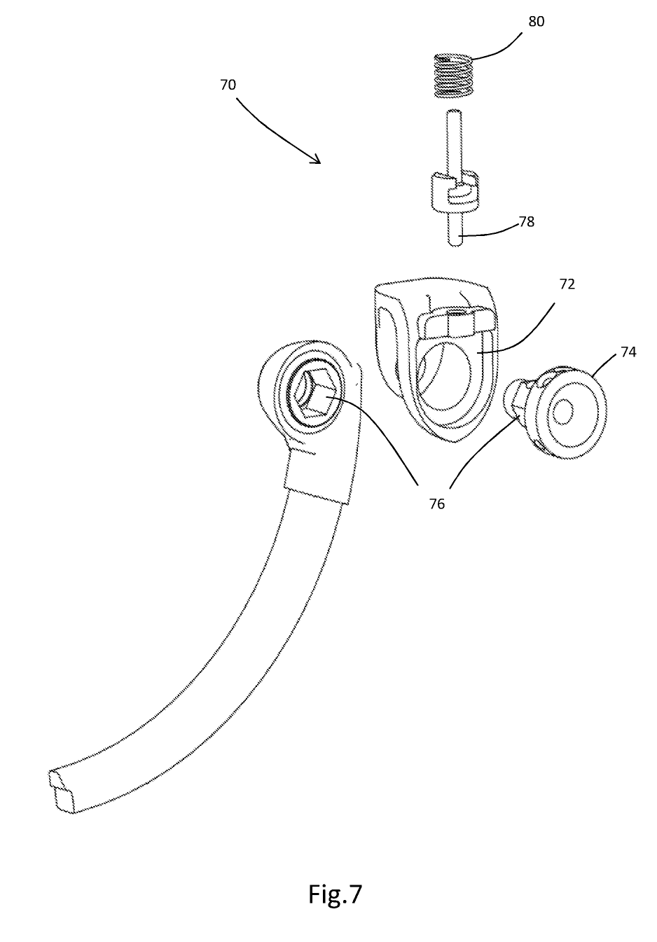

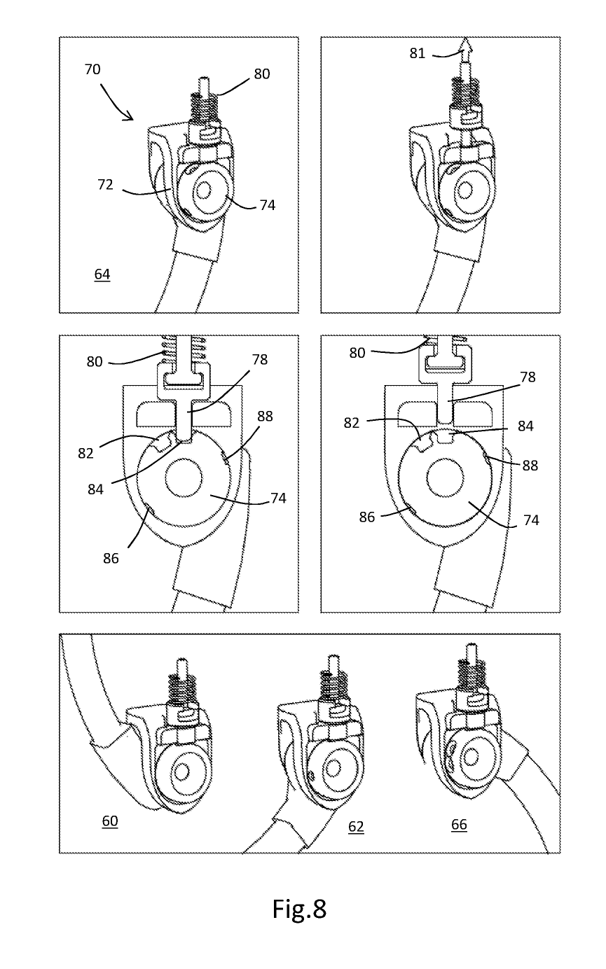

In a further alternative embodiment, the blade retaining member of the laryngoscope body has four positions, a storage position 60, a first working position 62, a second working position 64 and a third working position 66, as shown in FIGS. 7 and 8. The adjuster mechanism 70 comprises a socket 72, a drum 74 connected to the proximal end of the blade retaining member by a hexagonal engagement 76 and a locking pin 78. The drum comprises four apertures around the circumference of the drum for receiving the locking pin, corresponding to the four positions of the blade retaining member.

The locking pin is biased towards the drum by a spring 80 to retain the locking pin within an aperture once the locking pin has been received by the said aperture. The locking pin may be withdrawn 81 from an aperture by the user to move the blade retaining member to a new position.

When the locking pin is received by two of the apertures (82, 84) of the drum, the blade retaining member is locked into the first or second working position and these positions for use with direct view 82 and indirect view blades 84. The remaining two apertures of the drum are shallower than those for use with direct and indirect view blades to ensure that they do not fully engage the locking pin, and correspond to the storage position 86 and the third working position 88. As the said apertures do not fully engage the locking pin, whilst the blade retaining member is held in position, the user may manually move the blade retaining member from the storage or flexed position without releasing the pin, allowing the blade retaining member to be held initially in the third working position, useful for initially introducing a blade retained by the blade retaining member into the oral cavity of a patient where the chest of the patient restrict laryngoscope access. The locking pin then sits on the outer surface of the drum until the blade retaining member is returned to either the third working position or the second working position, whereupon the locking pin is received by the appropriate aperture and the blade retaining member position locked.

In a yet further alternative embodiment, the adjuster 90 connecting the blade retaining member to the elongate handle comprises a first plate 92 connected to the blade retaining member operable to move along an axis 94 within a limited range of movement, a second plate 96 connected to the elongate handle mounted within a socket 98 and coaxial with the first plate, a spring 100 biasing the first plate towards the second plate along the axis and a locking pin 102. The first plate and second plate comprise a series of teeth 104, 106 configured to engage each other when the first and second plates are in contact. The locking pin is biased towards the axis by a spring 108 such that the position of the first plate is fixed when the locking pin is engaged.

In a first position (e.g. a position suitable for use with a direct view blade), the teeth of the first and second plates are engaged and the locking pin is positioned to prevent the teeth of the first plate from disengaging the teeth of the second plate, such that the blade retaining member is locked in the first position. If a user wishes to move the blade retaining member to a second position (e.g. a position suitable for use with an indirect view blade), the locking pin is retracted to allow the first plate to be moved away from the second plate along the axis such that the teeth of the first plate disengage the teeth of the second plate. The first plate may then be rotated such that the blade retaining member is moved into the desired second position. The first plate is then allowed to move back towards the second plate, the teeth of the first plate engage the teeth of the second plate and the locking pin moves back to lock the position of the first plate.

In alternative embodiments the blades used for direct view and indirect view intubation procedures have the same curvature of the inferior surface adjacent to the channel of the blade but have a lifter portion extending at different angles relative to the tangent of the distal end of the channel, the direct view blade having a smaller angle and the indirect view blade having a greater angle.

Thus, a hospital may stock a kit of parts comprising a single laryngoscope body and at least two blades with different curvatures which are usable with the same laryngoscope, at least two of which are to be used with a different exit angle of the blade retaining member of the larnygoscope body.

In some embodiments, the blade retaining member is fixable in a plurality of discrete positions. In other embodiments, the blade retaining member can be fixed in a continuous range of positions. In an example embodiment, the blade retaining member can be fixed in first discrete position, at an exit angle of around 130-135° to retain a direct view blade but can also be fixed in a continuous range of positions, for example with an exit angle of between 140° and 180° to retain an indirect view blade. It may also be moved to an exit angle of up to 200° but not fixed beyond 180°. In the example embodiment, the blade retaining member is biased (e.g. by a first spring) to urge it to an increasing exit angle when the exit angle is less than the exit angle of the discrete position and biases to urge it to a reduced exit angle when the exit angle is greater than the exit angle of the discrete position (e.g. by the same spring or a second spring). However, a further spring biases the blade retaining member to increase the exit angle once the blade retaining member passes a defined exit angle, for example 180°. This means that although it will not be fixed, the blade can be conveniently located at the maximum possible exit angle 37 (which might for example be in the range 200-245°) and retained there by the biasing.

Further variations and modifications fall within the scope of the invention herein disclosed.

1. A laryngoscope body comprising an elongate handle and a blade retaining member configured to retain a blade such that the blade extends away from the elongate handle; wherein the orientation of the blade retaining member is variable to allow a blade to be retained on the blade retaining member at a plurality of angles relative to the elongate handle.

2. A laryngoscope body according to claim 1, wherein the blade retaining member defines the angle at which a blade retained on the blade retaining member extends from the handle.

3. A laryngoscope body according to claim 1, wherein the blade retaining member comprises an elongate member for slidably retaining a blade having a cooperating channel.

4. A laryngoscope body according to claim 1, wherein the blade retaining member is selectively deployable in a plurality of configurations in which in which the exit angle is restricted at least to the extent of limiting the maximum exit angle, wherein the maximum exit angle is different in each of the plurality of configurations.

5. A laryngoscope body according to claim 1, wherein the blade retaining member is selectively fixable in one, or a plurality of positions.

6. A laryngoscope body according to claim 5, wherein the blade retaining member is selectively fixable in a first position having a first exit angle, or has the maximum exit angle fixed at a first exit angle, suitable for use with a first blade having an inferior surface with a first curvature, and is also selectively fixable at a second position having a second exit angle, or having the maximum exit angle fixed at a second exit angle, which is greater than the first exit angle, suitable for use with a second blade having a inferior surface of with a second curvature which is greater than the first curvature.

7. A laryngoscope body according to claim 6, wherein the exit angle, or the maximum exit angle, is between 120 and 140 degrees in the first position.

8. A laryngoscope body according to claim 6, wherein the exit angle, or the maximum exit angle, is between 140 and 180 degrees.

9. A laryngoscope body according to claim 1, wherein the exit angle is adjustable to at least, or greater than 180 degrees and cannot be fixed beyond a maximum fixing exit angle.

10. A laryngoscope body according to claim 1, comprising a biasing mechanism to bias the exit angle between the blade retaining member and the handle.

11. A laryngoscope body according to claim 1, comprising a locking mechanism to lock the exit angle of the blade retaining member.

12. A laryngoscope body according to claim 1, wherein the laryngoscope comprises a resilient cover which extends at least over the flexible junction between the handle and the blade retaining member.

13. A laryngoscope body according to 1, wherein the laryngoscope body comprises a blade recognition element operable to recognise which type of blade is retained on the blade retaining member.

14. A laryngoscope body according to claim 13, wherein the laryngoscope body is configured to indicate to a user whether a blade retained by the blade retaining member is suitable for use in the current position of the blade retaining member upon recognition of the retained blade by the blade recognition element.

15. A laryngoscope body according to claim 13, wherein the laryngoscope body is configured to prevent use of a retained blade that is not suitable for use in the current position of the blade retaining member upon recognition of the retained blade by the blade recognition element.

16. A laryngoscope body according to claim 1, wherein the blade retaining member is movable automatically from a first position to a second position automatically.

17. A laryngoscope blade comprising a mounting element for mounting the laryngoscope blade to a blade retaining member and an electronically readable storage medium operable to store information relating to the type of the laryngoscope blade wherein the electronically readable storage medium is readable by the blade recognition element of the laryngoscope body according to claim 13.

18. A laryngoscope blade according to claim 17, wherein the electronically readable storage medium is a radio frequency identification tag.

19. A laryngoscope blade according to claim 17, wherein the information relating to the type of the laryngoscope blade is indicative of the curvature of the inferior surface of the laryngoscope blade.

20. A laryngoscope blade according to claim 17, wherein the mounting element is an elongate channel.

21. A kit of parts comprising a laryngoscope body according to claim 13, and a plurality of laryngoscope blades which comprise a mounting element for mounting the laryngoscope blade to the blade retaining member and an electronically readable storage medium operable to store information relating to the type of the laryngoscope blade, wherein the electronically readable storage medium of the laryngoscope blade is readable by the blade recognition element of the laryngoscope body, and wherein the plurality of laryngoscope blades comprises at least one laryngoscope blade having an inferior surface with a first curvature, and at least one laryngoscope blade having an inferior surface with a second curvature which is different to the first curvature.

22. A kit according to claim 21, wherein the at least one laryngoscope blade having an inferior surface with a first curvature is a direct view blade.

23. A kit according to claim 21, wherein the at least one laryngoscope blade having an inferior surface with a second curvature which is different to the first curvature is an indirect view blade.

24. A kit according to claim 23, wherein the at least one laryngoscope blade having an inferior surface with a second curvature is not suitable for use to directly view the larynx of a patient.

25. A kit according to claim 21, wherein the first curvature differs from the second curvature by at least 10 degrees.

26. A laryngoscope comprising a laryngoscope body according to claim 1, wherein the blade retaining member retains a blade, the blade extending away from the elongate handle, wherein the orientation of the blade retaining member is variable to vary the exit angle between the blade retaining member and the handle.

27. Apparatus comprising a laryngoscope body according to claim 1 and a plurality of blades, each blade in the plurality of blades comprising a mounting element for mounting the laryngoscope blade to the blade retaining member, the plurality of blades comprising a first blade having an inferior surface with a first curvature, and a second blade having an inferior surface with a second curvature which is different to the first curvature.

28. Apparatus according to claim 27, wherein the blade retaining member comprises an elongate member for slidably retaining a blade having a cooperating channel, the elongate member is fixable at a plurality of angles relative to the elongate handle and each of the blades within the plurality of blades comprise a said channel to receive the blade retaining member; the plurality of blades comprising two blades having inferior surfaces with a different curvature adjacent to the channel of each blade.

29. A method of using a laryngoscope comprising the steps of;
providing a laryngoscope body according to claim 1, a first blade and a second blade;
fixing the blade retaining formation in a first position having a first exit angle or deploying the blade retaining formation in a first configuration in which in which the exit angle is limited to a first maximum exit angle;
mounting the first blade to the blade retaining formation of the laryngoscope body;
using the laryngoscope body and first blade;
demounting the first blade;
fixing the blade retaining formation in a second position having a second exit angle which is different to the first exit angle, or deploying the blade retaining formation in a second configuration in which the exit angle is limited to a second maximum exit angle, which is different to the first maximum exit angle;
mounting the second blade to the blade retaining formation; and using the laryngoscope body and second blade.

30. A method of using a laryngoscope comprising the steps of:
providing a laryngoscope body according to claim 1 and a blade which comprise a mounting element for mounting the laryngoscope blade to the blade retaining member and an electronically readable storage medium operable to store information relating to the type of the laryngoscope blade, wherein the electronically readable storage medium of the laryngoscope blade is readable by the blade recognition element of the laryngoscope body;
fixing the blade retaining member of the laryngoscope body in a first position having a first exit angle or deploying the blade retaining member in a first configuration having a first maximum exit angle;
mounting the blade to the blade retaining member of the laryngoscope body;
inserting the blade into the oral cavity of a patient;
fixing the blade retaining member of the laryngoscope body to a second position having a second exit angle which is different to the first exit angle or deploying the blade retaining member in a second configuration having a second maximum exit angle which is different to the second exit angle; and
viewing the oral cavity, or a portion of the oral cavity of the patient.

31. A method according to claim 29, wherein the first blade is a direct view blade and the second blade is an indirect view blade, and the second exit angle or maximum exit angle is greater than the first exit angle or first maximum exit angle; or wherein the first blade is an indirect view blade, and the second blade is a direct view blade, and the second exit angle or maximum exit angle is less than the first exit angle or first maximum exit angle.

32. A method according to claim 30, wherein the first blade is a direct view blade and the second blade is an indirect view blade, and the second exit angle or maximum exit angle is greater than the first exit angle or first maximum exit angle; or wherein the first blade is an indirect view blade, and the second blade is a direct view blade, and the second exit angle or maximum exit angle is less than the first exit angle or first maximum exit angle.

33. A kit of parts according to claim 21, wherein the electronically readable storage medium is a radio frequency identification tag.

34. A kit of parts according to claim 21, wherein the information relating to the type of the laryngoscope blade is indicative of the curvature of the inferior surface of the laryngoscope blade.

35. A kit of parts according to claim 21, wherein the mounting element is an elongate channel.

36. A kit of parts according to claim 21, wherein the first blade is a direct view blade and the second blade is an indirect view blade, and the second exit angle or maximum exit angle is greater than the first exit angle or first maximum exit angle; or wherein the first blade is an indirect view blade, and the second blade is a direct view blade, and the second exit angle or maximum exit angle is less than the first exit angle or first maximum exit angle.

37. A method according to claim 30, wherein the electronically readable storage medium is a radio frequency identification tag.

38. A method according to claim 30, wherein the information relating to the type of the laryngoscope blade is indicative of the curvature of the inferior surface of the laryngoscope blade.

39. A method according to claim 30, wherein the mounting element is an elongate channel.
